(19) United States
(12) Patent Application Publication
(10) Pub. No.: US 2005/0129585 A1
(43) Pub. Date: Jun. 16, 2005

(54) Ozone-deodorizing rubbish container

(76) Inventor: Dai-Ming Kuo, Panchiao (TW)

Correspondence Address: Dai-Ming Kuo, P.O. Box 55-846, Taipei, 104, TW

(21) Appl. No.: 10/783,225

(22) Filed: Feb. 19, 2004

(30) Foreign Application Priority Data
Dec. 10, 2003 (TW) 92221721

(51) Int. Cl. A61L 2/20

(52) U.S. Cl. 422/123; 422/292; 422/186.12; 422/5

(57) ABSTRACT

An ozone-deodorizing rubbish container includes: an ozone generator installed in a rubbish (garbage or trash) container, whereby upon actuation of the ozone generator, the ozone gas as produced from the ozone generator will be applied into the interior in the rubbish container for efficiently deodorizing and disinfecting the stench gas in the rubbish container.

### BACKGROUND OF THE INVENTION

U.S. Pat. No. 6,365,113 to Jon L. Roberts disclosed a trash receptacle sterilization apparatus having ultraviolet sterilization in an enclosed container to kill bacteria and organisms.

However, such a prior art has the following drawbacks:
1. Many ultraviolet lamps (56, 58, 60, 62, 63) should be provided in the trash receptacle to increase installation complexity and maintenance problem for maintaining so many lamps within the container.
2. Air fans (36) should be provided to effect the circulation of ozone gas as produced from the ultraviolet lamps, thereby increasing production cost thereof.
3. Ozone gas is produced from ultraviolet lamps, having low yield of ozone as obtained from the ultraviolet radiation and thus affecting the deodorization efficiency for deodorizing the stench gas within the trash container.

The present inventor has found the drawbacks of the conventional trash container and invented the present ozone-deodorizing rubbish container.

### SUMMARY OF THE INVENTION

The object of the present invention is to provide an ozone-deodorizing rubbish container including: an ozone generator installed in a rubbish (garbage or trash) container, whereby upon actuation of the ozone generator, the ozone gas as produced from the ozone generator will be applied into the interior in the rubbish container for efficiently deodorizing and disinfecting the stench gas in the rubbish container.

### BRIEF DESCRIPTION OF THE DRAWINGS

FIG. 1 is a perspective view of the present invention.

FIG. 2 is a sectional drawing of the present invention.

FIG. 3 shows a circuit diagram of the ozone generator as provided in the present invention.

### DETAILED DESCRIPTION

As shown in the drawing figures, the present invention comprises: an ozone generator 2 installed in a rubbish (or garbage or trash) container 1.

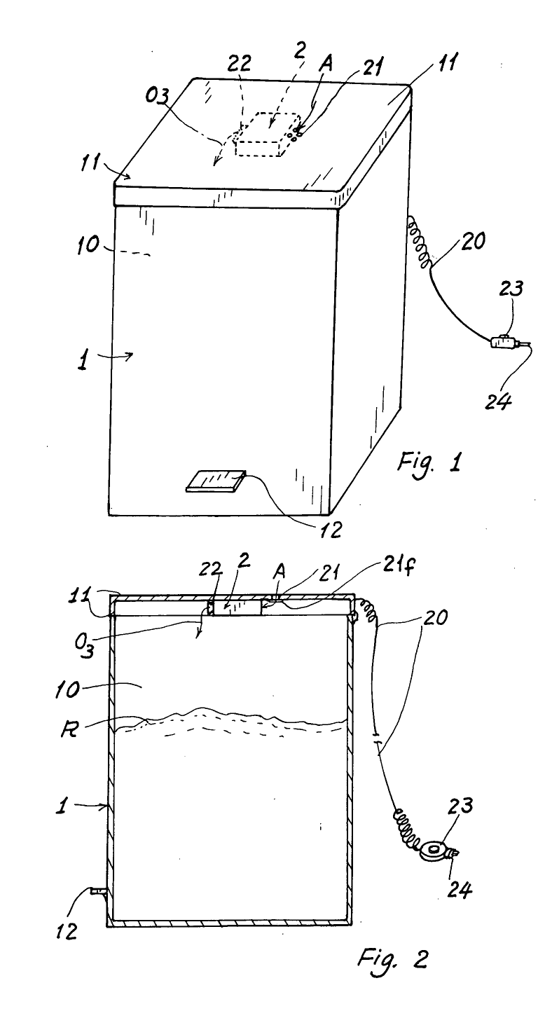

The ozone generator 2 may be provided in a top portion of the rubbish container 1 or may be fixed to a bottom portion or inside wall of a top cover 11 formed on a top of the rubbish container 1 as shown in FIGS. 1, 2.

At least an air inlet opening 21 is formed through the top cover 11 of the rubbish container 1 of the present invention for directing air inwardly into the container 1 to be partially converted into ozone O₃ by the ozone generator 2. An air-penetrable filter 21f such as made of active carbon is formed on the inside wall of the top cover 11 for preventing leakage of any stench gas from the opening 21 of the container 1.

Of course, upon opening of the cover 11 each time, the environmental air may enter the interior 10 within the container 1 for the conversion into ozone gas.

A gas outlet nozzle (or port) 22 is formed in the ozone generator 2 for discharging ozone gas O₃ into the interior 10 of the rubbish container filled with rubbish R therein.

An electric wire 20 is connected between the ozone generator 2 and an utility power supply or power supply source such as a plug 24 as shown in the figures.

A controller 23 for on-off control for switching on or off the power supply to the ozone generator 2 or for timing control for the actuation of the generator 2 is provided on the wire 20 or on other suitable location. Also, a timing controller (not shown) may be provided in the controller 23 for automatically actuating the ozone generator 2 for a pre-set time period or in each preset time interval for actuating the ozone generator 2 for producing ozone gas for deodorization and disinfection within the container 1.

The models of the ozone generator 2 as used in the present invention are not limited in this invention.

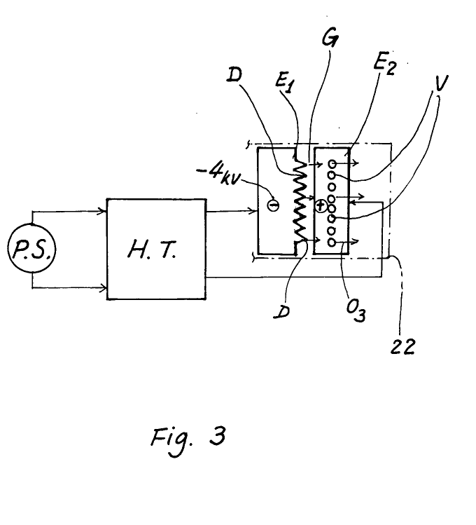

However, an ozone generator 2 having an electronic circuit diagram as shown in FIG. 3 may be preferably selected for use in the present invention, which will be further described in detail in the following description.

The locations for installing the ozone generator 2 within the rubbish container 1 are not limited. But, it is preferably installed at a top position within the rubbish container 1 or installed just beneath the top cover 11 of the rubbish container 1 since ozone is heavier than air and will gravitationally descend to effectively deodorize and disinfect the stench gas or air within the rubbish container.

For understanding the operation principles of the ozone generator 2, the circuit diagram as shown in FIG. 3 may be referred.

When the power source P.S. is closed to produce a high tension across the cathode E₁ and anode E₂ through the high-tension generator H.T., the air in the gap G (for example, 3 mm) between the two electrodes E₁, E₂ across the high-tension field will be ionized to form an “electronic wind” and the air is also partially converted into percentage of ozone O₃, which is then laden in the electronic wind caused by the ionized air and discharged downwardly into the interior 10 in the container 1 to deodorize or disinfect the stench gas or air in the rubbish container 1. The cathode E1 may be −4000 volts, while anode E₂ may be O volt (or other values of positive volts). A plurality of discharge tips D are juxtapositionally formed on the cathode E₁, each discharge tip D aiming at (or aligned with) each air vent V formed in the anode E₂. The air in the gap G will be ionized to form an electronic wind to flow from E₁ to E₂ and the air is also partially converted into ozone O₃ to be “blown” downwardly though the port 22 driven by the electronic wind (FIG. 3).

Since ozone O₃ is heavier than air, it will gravitationally descend for effective deodorization and disinfection within the container 1.

The air through the gap G in the ozone generator 2 may be converted from an insulator to be a semi-conductor under the high tension field to obtain a percentage of ozone, which may be driven from the first electrode E₁ having a plurality of discharge tips D towards the second electrode E₂ having a plurality of air vents V formed therein (as shown in FIG. 3). The ozone-containing gas may be driven through the vents V and then discharged into the interior 10 in the container 1 for deodorization and disinfection in the rubbish container 1.

The present invention is superior to any conventional ozone-effected trash or rubbish container with the following advantages:
1. A compact unit of ozone generator 2 is installed in the rubbish container 1 to thereby reduce the installation complexity and production cost; while increasing the maintenance convenience.
2. An ozone gas O₃ is directly quickly formed within the container to efficiently deodorize and disinfect the interior within the rubbish container without any delay for better hygienic effect.
3. No air fan is necessary in the container to thereby decrease the production cost and minimize the noise and electromagnetic wave interference as caused by a motor.

The present invention may be modified without departing from the spirit and scope of the present invention.

1. A rubbish container comprising:
an ozone generator formed in an upper portion of a rubbish container and operatively producing ozone gas directly in an interior in said rubbish container for deodorization and disinfection in said rubbish container.

2. A rubbish container according to claim 1, wherein said ozone generator is formed on an inside wall of a top cover of said rubbish container.

3. A rubbish container according to claim 1, wherein said ozone generator includes at least an air inlet opening in the container for directing air inwardly into said container, an outlet port formed in said ozone generator for discharging ozone gas as produced by said generator into said container, and a controller for on-off control and timing control for the actuation of said ozone generator.

4. A rubbish container according to claim 1, wherein said ozone generator includes a cathode electrically connected to a negative pole of a power source of high tension having a plurality of discharge tips juxtapositionally formed on said cathode, and an anode electrically connected to a positive pole of said power source and having a plurality of air vents each said air vent aligned with each said discharge tip, defining a gap between each said discharge tip of said cathode and each said air vent of said anode, whereby upon forming of a high voltage across said cathode and said anode to ionize air in said gap to form an electronic wind by ionized air to flow from said cathode to said anode and the air in the gap will be partially converted to ozone, which will be laden in and carried by said electronic wind to be discharged into said rubbish container for deodorization and disinfection in the container.
